(19) United States
(12) Patent Application Publication
(10) Pub. No.: US 2018/0198218 A1
(43) Pub. Date: Jul. 12, 2018

(54) ELECTRODE CABLE SYSTEM FOR NEUROLOGICAL MONITORING AND IMAGING

(71) Applicant: Rhythmlink International, LLC, Columbia, SC (US)

(72) Inventors: Shawn V. Regan, Columbia, SC (US); Leah L. Hanson, New Glarus, WI (US); Harrison Mr. Floyd, Columbia, SC (US); Paul R. Sharples, Cassatt, SC (US); Stacey L. Askelson, Duluth, MN (US); William H. Lewis, Lexington, SC (US); William W, Webb, Huntersville, NC (US); Daniel E. McCoy, Murrells Inlet, SC (US)

(73) Assignee: Rhythmlink International, LLC

(21) Appl. No.: 15/913,017

(22) Filed: Mar. 6, 2018

Related U.S. Application Data
(63) Continuation-in-part of application No. 14/537,064, filed on Nov. 10, 2014.

(51) Int. Cl. H01R 9/24; A61B 5/055

(52) U.S. Cl. CPC H01R 9/2475; A61B 2562/226; H01R 2201/12; A61B 5/055

(57) ABSTRACT

A cable management system is used in neurological monitoring and imaging procedures, particularly MRI. The system enables quickly disconnecting and quickly and accurately reconnecting the patient to the neurological monitoring equipment. The cable management system uses plural color-coded, two-part, cable pairs that include an electrode cable and an extension cable in each pair. The electrode and the short electrode cable remain attached to the patent during monitoring while the extension cable is disconnected and removed. A plug and socket combination that connects in a preselected orientation that enables the two parts to be quickly and accurately disconnected and reconnected. Metal is minimized in the electrode and electrode conductor in part by its short length, it thinness, the use of metal coating on plastic electrodes, and the use of alternative conductive materials.

### TECHNOLOGY FIELD

The present disclosure relates generally to neurological monitoring and, more specifically to electrodes for neurological monitoring in a magnetic resonance imaging environment.

### BACKGROUND

From time to time, patients whose brain activity is being monitored may need to undergo an imaging procedure such as magnetic resonance imaging (MRI) or computerized axial tomography (CAT) scanning.

Imaging procedures are non-invasive techniques for creating detailed information about the body to determine the presence and severity of certain injuries.

MRI equipment operates by moving a patient into a strong magnetic field. A strong magnetic field creates the magnetic resonance environment for excitation of atoms in cells of a patient's brain, which then emit photons that can used to generate images of the excited brain. Strong magnetic fields, in addition to making it possible to diagnose a patient's condition, may also have harmful side effects when certain objects, particularly those that include ferrous materials, enter these fields. Magnetic fields may trigger displacing forces and torques, radio frequency heating and the appearance of image artifacts on the MRI images. CAT scans, while not interacting with cells and objects in the same way MRI magnetic fields do, may still produce artifacts, or imperfections, in images when extraneous materials, particularly dense materials such as brass are scanned. Artifacts degrade the quality of the images produced and can interfere with reading the images.

To minimize these effects, materials and devices to be used in an imaging environment are carefully selected and controlled. Metal-containing devices, such as wire conductors, are generally avoided or their use minimized. Accordingly, when a patient undergoes an imaging procedure, the neurological monitoring electrodes previously attached to the patient's head are removed until after the procedure has been completed, and, when the patient is away from the imaging equipment, are the electrodes reconnected.

Electrodes have been developed that do not overheat in the strong magnetic field of MRI equipment and may be left in place during an MRI provided certain conditions and limitations of use are observed. However, the wires leading to them are disconnected from the electrodes. Leaving at least the electrodes in place during imaging simplifies reconnection of them to their lead wires and the resumption of monitoring.

However, further reductions in the time to resume monitoring would be desirable to make, because the unmonitored patient is vulnerable. While a patient is undergoing imaging, he or she is not being monitored. Reconnecting patients does not always begin when the patient is ready for monitoring to resume. Those qualified to attach lead wires to electrodes and attach electrodes to a patient's scalp are often in short supply and a patient will wait until someone is available to reconnect the lead wires. While waiting for their electrodes to be reattached to the neurological monitoring equipment or to have a new set of electrodes attached, these patients may suffer certain types of injuries that have no outward signs and which could be detected if they were being monitored.

Attaching and removing a set of electrodes may take an additional 20 minutes or more if a technician is available. Moreover, whenever electrodes and lead wires are removed or attached, there is the possibility of connection errors and increased risk of injury via skin breakdown underneath the electrode sites. The error may be merely an inconvenience or it may have serious consequences. Finally, operating room and hospital room time has a cost even if the patient is just waiting to have electrodes connected or disconnected. Essentially, there needs to be a better way to provide imaging for patients undergoing neurological monitoring.

### SUMMARY

The present cable system is for use in neurological monitoring that is especially suited for patients who may also need to undergo imaging procedures, particularly MRI and CAT scans. The present cable system makes it possible to disconnect the monitored patient quickly from, and to reconnect the patient quickly and accurately to, the neurological monitoring equipment. The present cable system facilitates connection and reconnection of the patient by (1) using a set of color-coded, two-part, cable pairs that include an electrode cable with an electrode that remains attached to the patient throughout an imaging procedure and an extension cable that is disconnected from the electrode cable prior to an imaging procedure. Both parts of the cable are pre-labeled to assist a radiological technician in mapping the component parts of the cable system to the patient's scalp. A connector on the ends of the electrode cables and another connector on the ends of the extension cables enables the electrode cables to be connected to the extension cables correctly.

Those familiar with neurological monitoring will appreciate these and other features and advantages of the cable system from a careful reading of the embodiments described herein, accompanied by the following drawings.

### BRIEF DESCRIPTION OF THE DRAWINGS

In the figures,

FIG. 1 is a perspective view of a single cable pair for an imaging procedure, according to an aspect of the present cable management system;

FIG. 2 is a perspective view of a cable management systems, ready for use with a cable pair of the set similar to the pair shown in FIG. 1;

FIG. 3 is a perspective view of a portion of the cable management set of FIG. 2 with the electrode cables shown separated from the extension cables, according to an aspect of the disclosure;

FIG. 4 shows a modular connector socket on the end of a cable of plural extension cables in the form of a ribbon cable;

FIG. 5 illustrates a cable management system, according to an alternative aspect of the disclosure;

FIG. 6A, 6B, and 6C show a perspective views the plug and socket connectors for the alternative cable management system of FIG. 5, shown separated in FIG. 6A, shown in the unlocked position and being joined in FIG. 6B, and shown in the locked and joined position in FIG. 6C, according to an aspect of the disclosure;

FIG. 7 illustrates a cross sectional view of the extension cable with mesh sleeve, according to an aspect of the disclosure; and

FIG. 8 is a flow diagram showing the series of steps for using the present cable set, according to an aspect of the disclosure.

### DETAILED DESCRIPTION

The present cable management system may be used with electrodes intended for sensing electroencephalographic (EEG), electromyographic (EMG), and evoked potentials, or as a ground cable for these types of measurements. As used herein, the term “set” means an electrode cable together with an extension cable. A cable pair 10 includes a particular electrode conductor and its corresponding extension conductor. The present cable management system includes at least one cable pair 10.

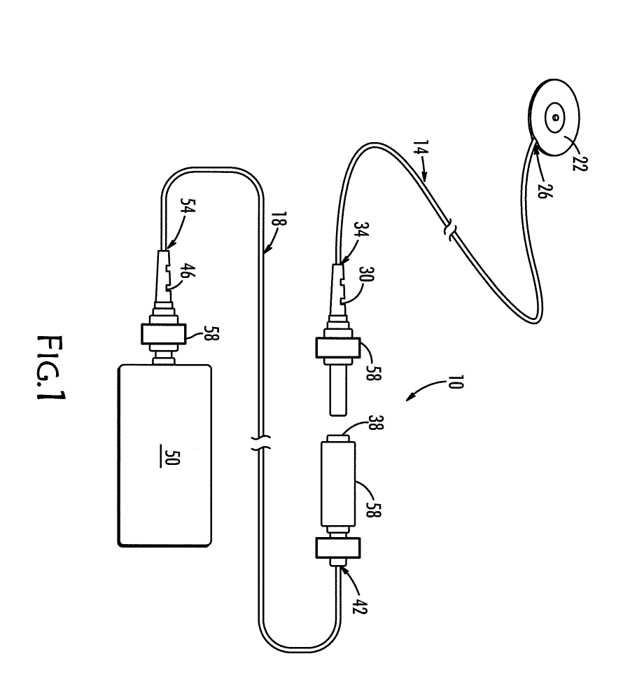

As shown in FIG. 1, a cable pair 10 has two parts: the electrode conductor 14 and the extension conductor 18. The two parts are joined to make cable pair 10 and the two parts of which are separable into the shorter electrode conductor 14 and a longer extension conductor 18. The electrode conductor 14 may carry an electrode 22 on a first end 26 and a connector 30 on the opposing second end 34 of electrode conductor.

Extension conductor 18 carries a connector 38 on a first end 42 that mates with connector 30 and another connector 46 on an opposing, second end 54 for plugging into an amplifier 50 for neurological monitoring. Connectors 30 and 38 on second end 34 of electrode conductor 14 and first end 42 of extension conductor 18, respectively, connect in any mechanical way, such as by male/female configuration fit. Connectors 38, 46, on first end 42 and second end 54 of extension conductor 18 may also conveniently comprise a male-female pair because connector 30 on second end 34 of electrode conductor 14 may be dimensioned and configured to be plugged directly into amplifier 50. Accordingly, connectors 30 and 46 may have the same configuration.

Electrode conductor 14 is much shorter than extension conductor 18. Electrode conductor 14 is long enough to able to just clear the hair of the patient.

For example, a suitable length for electrode conductor 14 is 24 cm (10 inches) or less; a suitable length for extension conductor 18 is at least 1 m. The length of cable pair 10 (an electrode conductor 14 and an extension conductor 18 combined) is conveniently long enough to span the distance between the head of the patient and amplifier 50 to which cable pair 10 is to be connected. If a greater distance exists between amplifier 50 and the head of the patient, a longer extension conductor 18 may be chosen than a longer electrode conductor 14.

Electrode 22 on first end 26 of electrode conductor 14 may be a cup electrode, a hydrogel electrode, needle electrode, surface electrode, or other electrode for attaching to the patient's skin, in the skin, or under the skin, and which electrode is in turn attached to the second end electrode conductor.

For MRI imaging, electrode 22 may be selected that is more suitable for use in an imaging environment. Electrode 22 is attached to electrode conductor 14 using an electrically conducting adhesive, solder, weldment, heat stake, or crimped tab to attach electrode 22 and electrode conductor 14 together. In order to avoid MRI artifacts on the image, the electrically conductive adhesive or a small clip would be preferable. Similarly, because electrode conductor 14 is shorter than extension conductor 18, it can be made of a different material, such as a material that is better in an imaging environment, for example, thinner metal wire, a non-metal conductor such as carbon fiber wire, or a different metal such as copper that has not been tinned. These different materials will respond in varying degrees to an imaging environment based on material and mass, and to the extent that certain materials may cost more for better performance in those environments, the shorter length in electrode conductor 14 offsets at least part of the cost differential.

Electrodes 22 may be inexpensive enough for single use, so they can be disposed of safely and economically after use. For example, electrodes 22 may be made of ABS plastic coated with metal such as a mixture of silver and silver chloride or of gold with electrode conductor 14 insulation made of vinyl or other customary insulating materials. It may also be possible to reuse the extension conductors while disposing of the electrode conductors, providing further economy.

For neurological monitoring, electrode 22 is attached to the scalp of the patient, either on, in, or under the skin depending on the choice of electrode. Electrode 22 may carry an anatomical descriptor to facilitate use, that is, a number, an alphanumeric symbol, or an icon that informs the user of the place on a patient's scalp where it is to be attached. For example, the well-known 10-20 International System is a set of locations on the scalp for placement of electrodes in the context of EEG tests. A position is defined by a letter that indicates the lobe or the letter C to indicate the center between lobes and the letter combination Cz to indicate the top center of the head, and also a number to indicate positions within a lobe. Often harnesses are used with pre-designated places for scalp electrodes. These harnesses typically use the 10-20 system.

Electrode 22 may carry that number in addition to or alternatively to the symbol described above to signify that electrode 22 is to be attached to the corresponding location. If electrodes 22 are attached to the patient with a conductive paste such as, for example, WEAVER Ten20 Conductive Paste manufactured and sold by Weaver and Company of Aurora, Colorado. Then connector 30 on second end 34 of electrode conductor 14 is connected to connector 38 on the first end 42 of extension conductor 18. Second end 54 of extension conductor 18 is then connected to amplifier 50.

When the patient is to undergo an imaging procedure, electrode 22 will remain in place on the patient's scalp. Connectors 30, 38 joining electrode conductor 14 and extension conductor 18 are separated so that extension conductor 18 may be removed from and kept away from the magnetic resonance environment. After the imaging procedure is completed, and the patient removed from the imaging area, connectors 30, 38 of electrode conductor 14 and extension conductor 18 can be reconnected and the neurological monitoring resumed.

The short length of electrode conductor 14 is sufficiently short so that it does not pose a significant risk to the patient when its electrode 22 remains attached to the patient's scalp while the patient is in an imaging environment. In that environment, neurological monitoring data is not be collected.

Use of electrode conductors 14 in a magnetic resonance environment is limited in time, for example, to 15 minutes, and field gradient strength, such as 4000 Gauss/em (or 40 T/m). Under these conditions, prior art conductors may increase in temperature by 2° C. In 15 minutes with a 1.5 Tesla system, the temperature of electrode conductor 14 may rise from electromagnetic heating not more than 0.9° C. with a background temperature increase of 0.7° C. In a 15-minute test with a 3.0 Tesla system, a temperature rise of not more than 1.5° C. against a background temperature increase of 0.6° C., has been detected in electrode conductor 14.

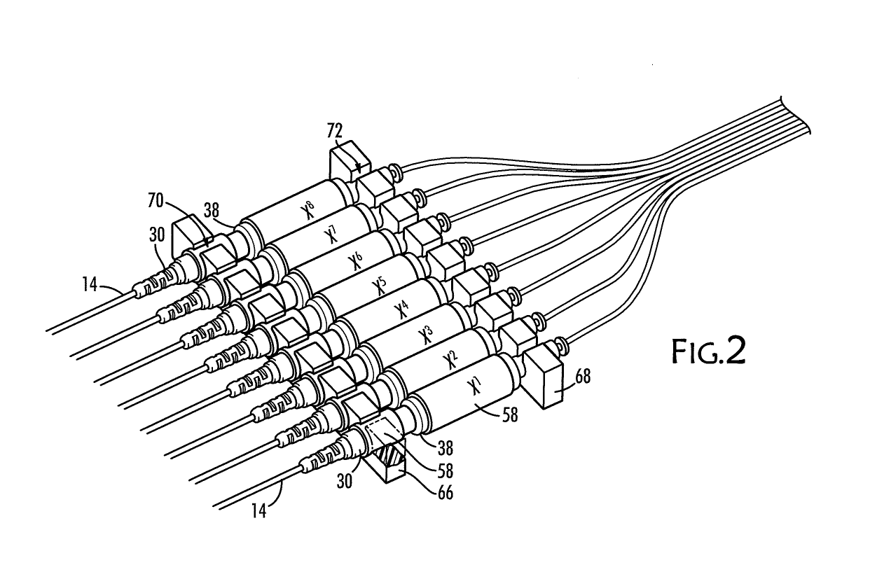

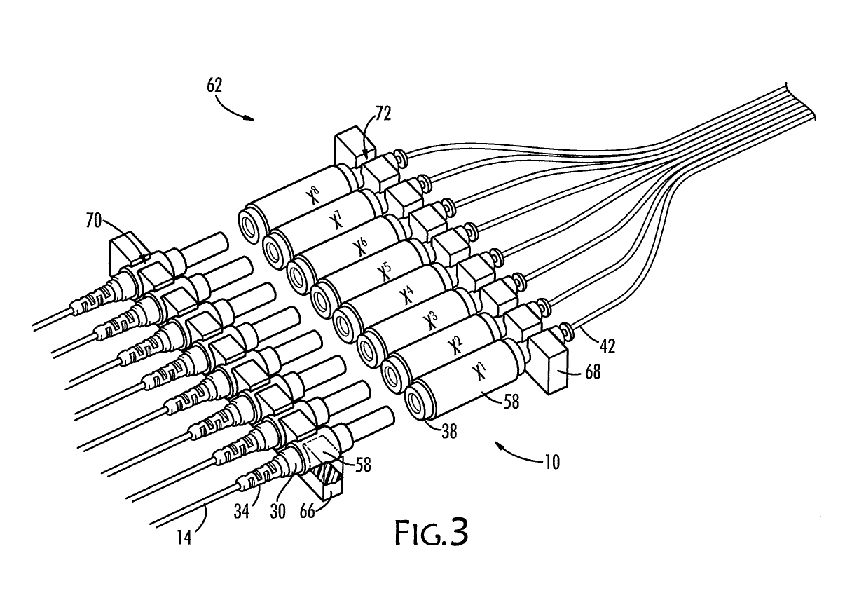

Because of the number of cable pairs 10 used in neurological monitoring may be as many as 48 cable pairs per patient or even more, there is a need to connect and reconnect the pairs safely, properly, and efficiently, and to know to what position on the patient's scalp they are attached. Moreover, these cable pairs 10 may be connected for monitoring and then disconnected numerous times for imaging procedures when the patient will not be monitored. Disconnecting and reconnecting cable pairs 10 take time away from the time for monitoring, so speed with accuracy is important. Therefore, to assure accurate and efficient connection and reconnection, plural cable pairs 10, as seen in FIGS. 2 and 3, may be coded using one color for both the electrode and extension conductor insulation and another one for the connectors attached to them to define a color pairing.

Alternatively or in addition, alpha-numeric characters or indicia may be used for redundant confirmation that a particular electrode conductor and extension conductor comprise a cable pair. Importantly, the particular electrode conductor and extension conductor is readily, and ideally redundantly, associable as a pair intended for connecting, and to be designated for use as a pair in obtaining data from a particular part of the patient's brain. Finally, the present cable pair may be marked so that a conductor of the pair is immediately identifiable as acceptable, conditionally acceptable, or unacceptable for use in an imaging environment.

Typically, cabling insulation is made in 13 colors, including red, green, dark blue, yellow, black, white, orange, grey, light blue, purple, brown, pink and light green. An additional code element can be conveniently added by a selecting a color for connectors 30, 38, 46, such as white, black, red, green and blue. For redundancy in identifying conductors, connectors 30, 38, 46, in that cable pair 10 may be identified by, for example, having bands 58 heat shrunk onto them with a number ranging from 1-48 or other alpha-numeric indicia that corresponds to a particular color combination of electrode conductor 14 and extension conductor 18. For example, an electrode conductor 14 and extension conductor 18 may both be insulated in yellow and have the same number 7 on bands 58 of their connectors 30, 38, 46 in the color red; alternatively, electrode conductor 14 and extension conductor 18 may be insulated in the color orange and with connectors 30, 38, 46, in the color red and with the number 8 on bands 58 on connectors 30, 38, 46. Alternatively, other ways to mark the bands could be used including stamping, pad printing, laser etching, laser printing, or injection molding of alpha-numeric indicia.

The twelve colors for conductor insulation and four colors selected for connectors yields a total number of pairings of 48. Of course, additional colors or other indicia for both the conductors and connectors increase the number of possibilities.

In addition, the bands may also be marked in some convenient way to signify a conductor that may be used unconditionally in the magnetic field of an MRI procedure, or used subject to conditions, and a conductor that may not be used in a magnetic field. As an example, yellow may signify an MRI conditional conductor and red may signify an MRI unacceptable conductor. The band may also carry a symbol known for the same messages in addition to a conductor color: either conditional for MRI use or not acceptable for MRI use.

A set of extension conductors 18 may be formed as a ribbon cable 62, a braided cable or as separate conductors. A ribbon cable 62, as shown in FIGS. 1 and 2 is a set of wires that are lying in parallel and in the same plane, separated slightly, and sharing insulation or that are held together in that configuration in some other way to form a flat “ribbon” composed of the plural wires of the set. Electrode conductors 14 are more likely separated (or at least separable) and have insulation in colors corresponding to the color of the insulation of extension conductors 18 so that, when joined with extension conductors 18, cable pair 10 is insulated in the same color, and which is a different color from that of any other cable pair 10.

Second end 34 of the electrode conductor 14 and first end 42 of extension conductor 18, which are separated prior to the MRI procedure and re-connected afterwards, may carry a band 58 containing an indicia that identifies the scalp location for attachment. Electrode 22 may be attached to the scalp of the patient in a specific location on the patient's head, and which location may be defined by the 10-20 International Standard. The data obtained from electrode 22 is location-specific and are combined with the data of other electrodes 22 in other locations on the patient's scalp to produce a composite image of brain activity from the collected neurological monitoring data traces. Accordingly, knowing which second end 54 of an extension conductor 18 is connected to which position on the head of a patient is of interest. Color-coding of electrode conductor 14 and extension conductor 18, helps to assure that electrode conductor 14 is connected correctly to extension conductor 18 and it does not tell where on the patient's head cable pair 10 is to be attached.

To specify an electrical path from amplifier 50 to head location to pass neurological monitoring signals, cable pair 10 is also marked with bands 58 with a shorthand indicator of the specific location it is associated, such as the 10-20 International Standard designators described above. An identifier made of two digits or letters is a simple way to establish a correspondence between cable pair 10 and the location on the patient's scalp. The identifier may be printed on a band 58 made of plastic that is applied to connectors 30, 38, 46, on cable pair 10, for example, by heat shrinking them. Three bands 58, one on connectors 30, 38, 46, of cable pair 10 may carry the same identifier and be presented in, say, the color red on band 58 on the connectors 38, 46 of the extension conductor 18 to signify the extension conductor 18 is not MRI acceptable and in yellow on band 58 on connector 30 of electrode conductor 14 to signify it is conditionally MRI acceptable.

Electrode conductor 14 has a length suitable for use in an imaging environment, that is, its length is short enough to minimize the effects of the magnetic fields that it is exposed to in the imaging environment in the limited time the imaging procedure takes to be completed. Extension conductor 18 has to be long enough to reach from near the patient to amplifier 50 and would be too long to use in an imaging environment because it's use may result in radio frequency heating or image side effects when in that environment during the time imaging procedures take. Therefore, extension conductor 18 is disconnected from electrode conductor 14 prior to the patient entering the imaging environment for imaging procedures. Electrode conductor 14, on the other hand, may remain attached to the patient despite the procedure because the effects of the procedure on it and vice versa are not significant.

The cable management system may also include a first cable lock 66 and a second cable lock 68. First cable lock 66 and second cable lock 69 may be configured for use as holders for the electrode conductors 14 and extension conductors 18. Second cable lock 68 may be used for extension conductors 18. First cable lock 66 may be used for holding the electrode conductors 14. Using first cable lock 66 and second cable lock 68, enables the user to separate plural cable pairs 10 by simply separating first cable locks 66 from second cable lock 68. Other types of locks or holders would also be suitable so long as they hold cable pairs 10 in an order so that electrode conductors 14 and extension conductors 18 of cable pair 10 can be quickly and accurately separated and reattached. Modular connector sockets such as an RJ-45, or RJ-50 may simplify the process of connecting cable pairs 10 together and serve as alternate embodiments of first cable locks 66 and second cable 68.

First cable lock 66 and second cable 68 have a number of grooves 70, 72, respectively, formed therein that at least matches the number of cable pairs 10, as seen in FIGS. 2 and 3. Connector 30 on the second end 34 of electrode conductor 14 is inserted into a groove 74 of first cable lock 66, and connector 38 of first end 42 of extension conductor 18 in cable pair 10 is inserted into a groove 78 of second cable lock 68. If first cable lock 66 and second cable 68, holding the set of connectors 30, 38, are moved towards each other and aligned laterally, connectors 30 on second end 34 of electrode conductors 14 and connectors 38 on first end 42 of the extension conductors 18 will be aligned and can easily be joined. The colors of electrode conductor 14 and extension conductor 18 of cable pair 10 will be visible and will match when properly sorted. Discrepancies in the sort are readily visible when the conductors are side-by-side. Furthermore, indicia on bands 58 carried on connector 30, connector 38, and connector 46, of the electrode conductor 14 and extension conductor 18, are the same.

Separating electrode conductors 14 from extension conductors 18 for the MRI procedure is simplified by using first cable lock 66 and second cable lock 68, while maintaining alignment of electrode conductors 14 and extension conductors 18, for when they need to be rejoined.

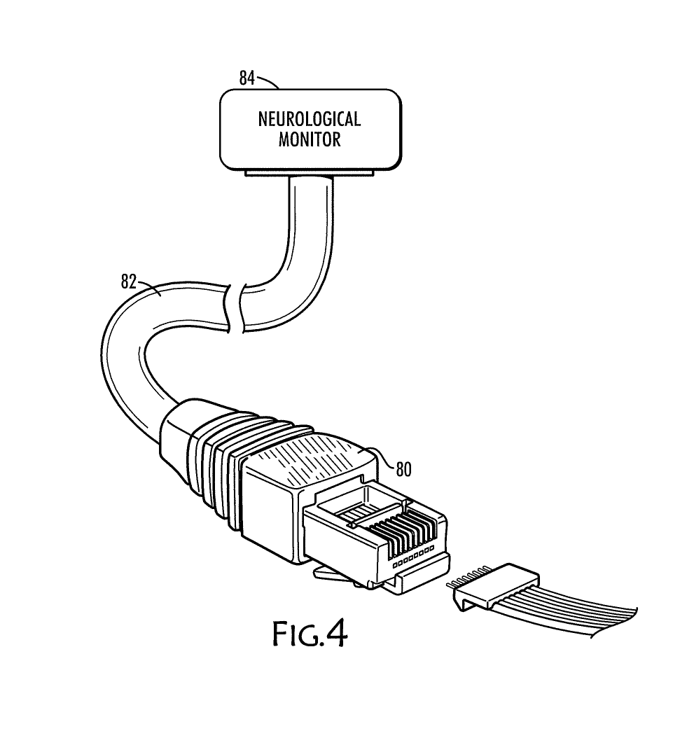

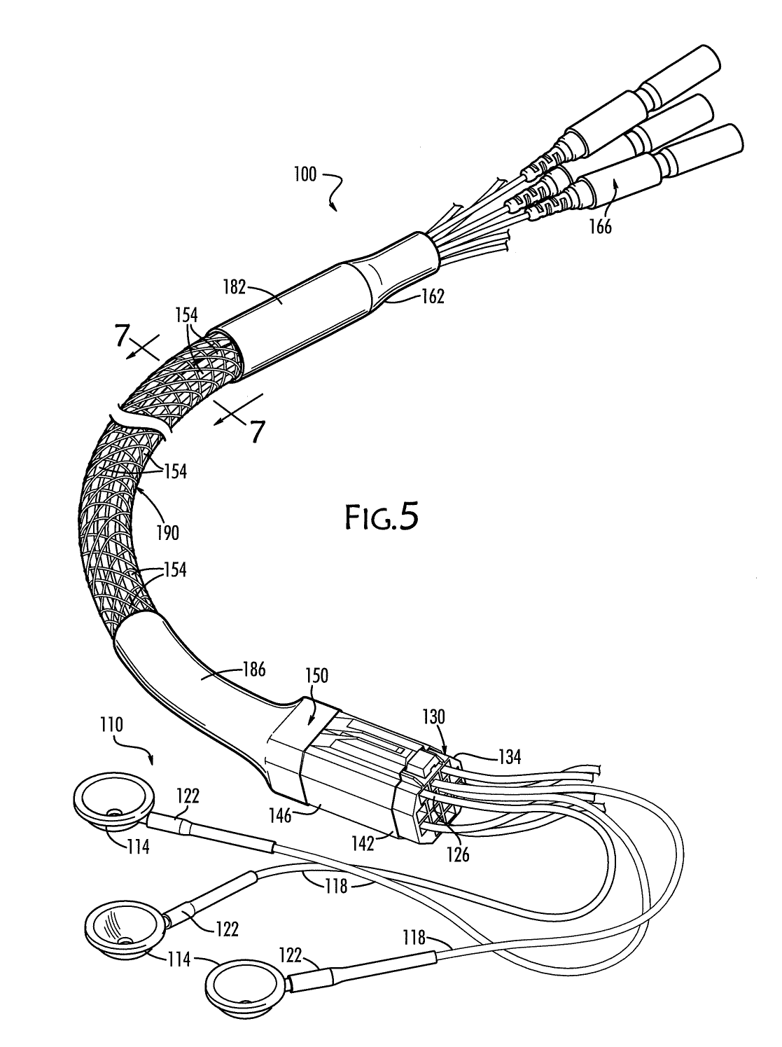

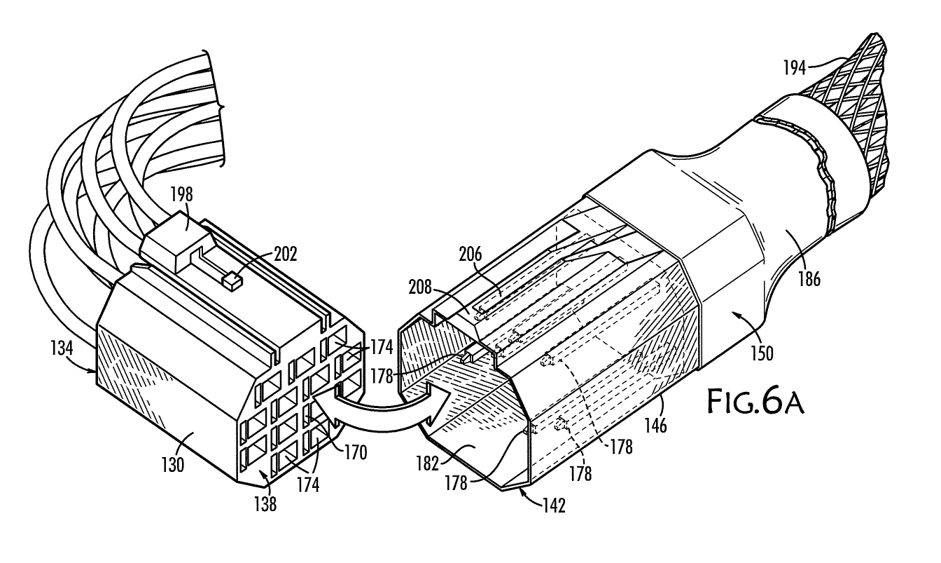

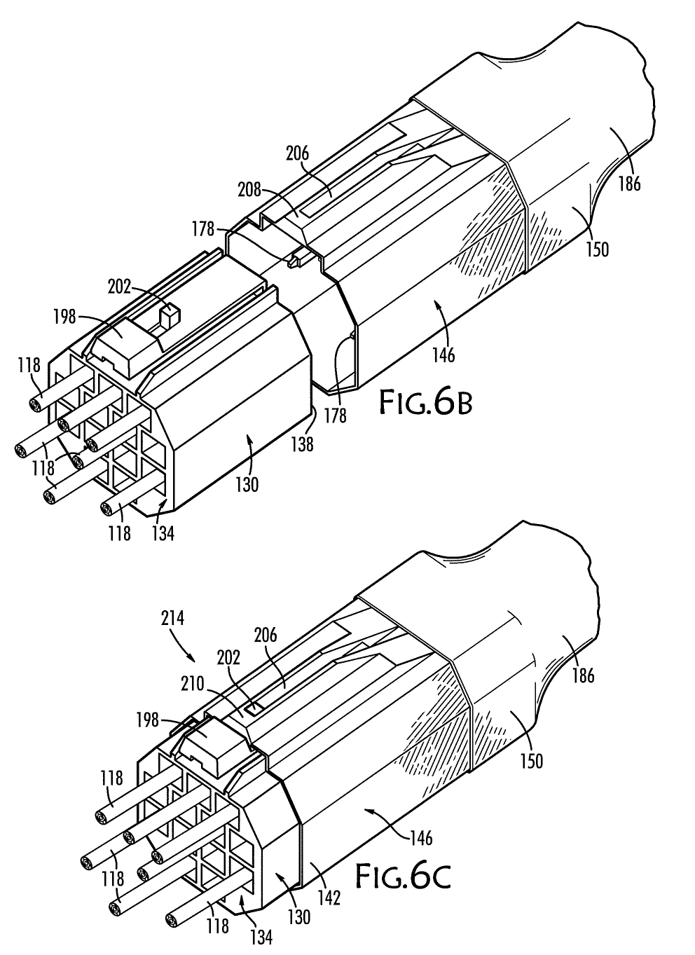

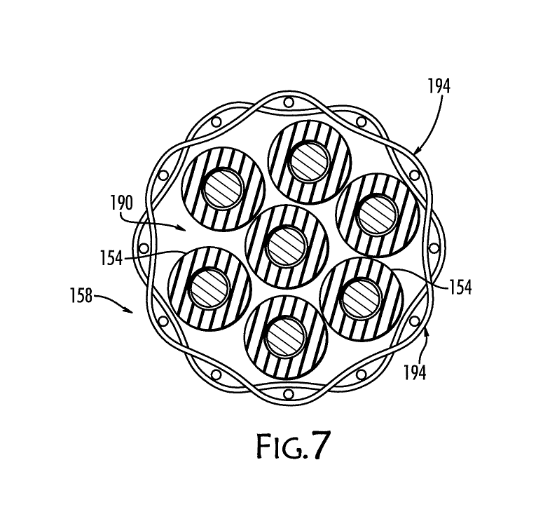

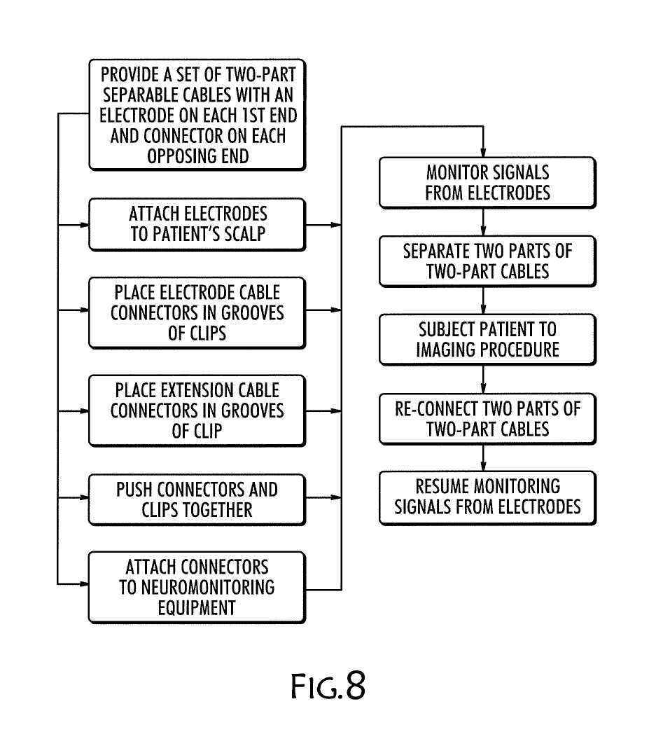

In an alternative example of a two-part electrode cable management system 100, shown in FIGS. 5 and 6A-6C, an electrode set 110 includes plural electrodes 114 (best seen in FIG. 5). An electrode 114 is electrically connected to an electrode conductor 118. Electrode conductor 118 has a first end 122, to which electrode 114 is connected, and electrode conductor 118 has a second end 126. Second end 126 of electrode conductor 118 is connected to a plug 130. Plug 130 has a first end 134 and a second end 138 (see FIG. 6A). Second end 126 of electrode conductor 118 is received inside a first end 142 of a socket 146. Plug 130 is shown partially inserted in socket 146 in FIG. 6B and fully inserted in socket 146 in FIGS. 5 and FIG. 6C. Socket 146 has a first end 142 and a second end 150. Second end 150 of socket 146 is electrically connected to a first end 154 of extension conductor 158. Extension conductor 158 of the extension conductors 158 has a second end 162 that is best seen in FIG. 5. Second end 162 of extension conductor 158 is electrically connected to connectors 166, best seen in FIG, 5, and which connector 166 is similar to connector 46 as shown in FIG. 1. Connector 166 may be plugged into a neurological monitor 84 (as shown in FIG. 4).

Accordingly, an electrical signal detected by electrode 114 travels from first end 122 of electrode conductor 118 to second end 126 and into first end 134 of plug 130 and then across plug 130 to second end 138 and next into first end 142 of socket 146. From second end 150 of socket 146, the electrical signal travels from first end 154 of extension conductor 158 and then to second end 162 and thence into a connector 166. When connector 166 is inserted in to a neurological monitor 84, the electrical signal from electrodes 114 can be processed.

The first end 134 of plug 130 has plural first contacts 170, best seen at second end 138 of plug 130, one first contact 170 for each electrode 114 of electrode set 110. Second end 138 of plug 130 contains plural receptacles 174, which receive a pin 178 from first end 142 of socket 146. Pin 178 is received in receptacle 174. Pin 178 is recessed in a shroud 182 to protect it from damage. Shroud 182 is configured to facilitate alignment of pin 178 with receptacles 174.

Pin 178 of socket 146 is in electrical connection with first end 154 of extension conductor 158. A band 186 holds second end 150 of socket 146 to first end 154 of extension conductor 158 in socket 146 and gathers plural extension conductors 158 into a bundle 190.

To facilitate handling of bundle 190 and to prevent individual extension conductors 154 from being pulled loose from bundle 190, a mesh sleeve 194 surrounds bundle 190. Mesh sleeve 194 may be a polymeric mesh sleeve or other plastic that is flexible and slippery (not likely to adhere to other materials) so as to facilitate handling, avoid snagging, and glide easily over surfaces encountered.

Plug 130 and socket 146 may lock together and are formed so that they can be pushed together if they are in a preselected orientation. That preselected orientation assures that electrode conductors 118 will be connected with extension conductors 158 in a pre-selected order.

In particular, to lock plug 130 and socket 146 together, a lever 198 with a tooth 202 on its end is carried on the surface of plug 130. A channel 206 dimensioned to receive lever 198 and a bridge 208 over channel 206 is formed in shroud 182 of socket 146. When plug 130 is inserted into socket 146, lever 198 allows tooth 202 to slide into channel 206 under bridge 208. When plug 130 is inserted far enough into socket 146, tooth 202 is cammed down by bridge 208 until plug 130 is fully seated in socket 146, at which point, tooth 202 springs back and into engagement with the end of bridge 208, which prevents removal of plug 130 from socket 146. Pressing down on lever 198 and beginning to remove plug 130 from socket 146 moves tooth 202 under bridge 208 where it cannot block separation of plug 130 from socket 146 whereupon plug 130 and socket 146 can be separated.

Accordingly, an electrode set 110 that includes plural electrodes 114 at one end to be attached to the head of a patient and plural connectors 210 at the other end to be plugged into an neurological monitor 84 for monitoring. When an imaging procedure is to be performed on the patient, the user disconnects the plug 130 from the socket 146 by pressing down on the tooth 202 until it no long locks the plug 130 and socket 146 together, and then pulls them apart, leaving extension . Electrode 114 and electrode conductors 118 remain with the patient during the imaging procedure while electrode conductor is removed from the patient. When the procedure is finished, plug 130 and socket 146 are pushed together so they lock and monitoring may resume.

In use, as shown in FIG. 9, the user attaches electrodes 114 on the first ends 122 of electrode conductors 118 to the scalp of the patient in the correct locations for neurological monitoring. Second ends 126 of electrode conductors 118 terminate in receptacles 174 inside plug 130, and first end 154 of extension conductors 158 terminate in pins 178 within a shroud 182 of socket 146. Therefore, to connect electrode conductors 118 to extension conductors 158, plug 130 and socket 146 are oriented so as to fit together and then plug 130 is inserted into socket 146 until the pins 178 enter receptacles 174, whereupon electrical contact is made and plug 130 and socket 146 lock together. Connectors 166 on second ends 162 of the extension conductors 158 are plugged into a neurological monitor 84 for monitoring to commence.

When an MRI procedure is required, the plug 130 and socket 146 of the two-parts of two-part cable management system 100 are separated by releasing plug 130 from socket 146 by simply depressing the tooth on the lever of the plug so that it slides under the bridge on socket. Plug 130 may then be pulled free of socket 146 to separate electrode set 110 from extension conductor 158. Then, the patient undergoes the MRI procedure. To re-connect the electrode set 110 to extension conductor 158, the process is reversed and monitoring then may resume.

Based on the foregoing, those skilled in the art of neurological monitoring will understand that many modifications and substitutions may be made in the foregoing embodiments without departing from the spirit and scope of the present disclosure, which is defined by the appended claims.

What is claimed is:

1. A device, comprising:
(a) a neurological monitoring electrode;
(b) an electrode conductor having a first end and a second end, said first end of said electrode conductor being connected to said neurological monitoring electrode;
(c) a plug having a first end and a second end;
(d) a receptacle within said plug, said second end of said electrode conductor being held by said receptacle within said plug;
(e) a socket having a first end and a second end, said socket having a pin, said first end of said socket receiving said second end of said plug, said pin being in electrical connection with said second end of said electrode conductor in said receptacle;
(f) an extension conductor having a first end and a second end, said first end of said extension conductor being electrically connected to said pin of said socket; and
(g) a connector connected to said second end of said extension conductor, said neurological monitoring electrode, said electrode conductor, said pin, said extension conductor and said connector defining an electrical path operable to pass neurological monitoring signals.

2. The device of claim 1, further comprising a sleeve surrounding said extension conductor.

3. The device of claim 1, further comprising a mesh sleeve surrounding said extension conductor.

4. The device of claim 1, further comprising a polymeric mesh sleeve surrounding said extension conductor.

5. The device of claim 1, wherein said socket and said plug lock together when said plug is received by said socket.

6. The device of claim 1, wherein said plug carries a lever, and wherein said socket has a channel dimensioned to receive said lever, said channel having a bridge thereover, said lever moving into said channel when said plug is received by said socket, said lever being cammed by said bridge when said lever moves into said channel past said bridge and is not cammed when said lever has moved past said bridge, said plug being in a locked position when in said channel and past said bridge and in an unlocked position when not past said bridge.

7. The device of claim 1, wherein said socket and said plug are operable to fit together in a preselected orientation.

8. The device of claim 1, wherein said socket has a shroud and wherein said plug fits into said shroud in a preselected orientation.

9. The device of claim 1, wherein said neurological monitoring electrode is crimped to said electrode conductor.

10. The device of claim 1, wherein said neurological monitoring electrode attached to said electrode conductor by a conductive adhesive.

11. The device of claim 1, wherein said neurological monitoring electrode attached to said electrode conductor by a heat stake.

12. The device of claim 1, wherein said electrode conductor is made of a different material than said extension conductor.

13. The device of claim 1, wherein said electrode conductor is not made of metal.

14. The device of claim 1, wherein said electrode conductor is made of carbon fiber.

15. The device of claim 1, wherein said electrode conductor comprises a first metal wire and said extension conductor comprises a second metal wire, and wherein said first metal wire is thinner than said second metal wire.

16. The device of claim 1, wherein said first end of said electrode conductor carries first indicia and said second end of said extension conductor carries second indicia and wherein said first indicia and said second indicia correspond.

17. The device of claim 16, wherein said first indicia has a number and said second indicia has said number.

18. The device of claim 1, wherein said electrode conductor is made of a first metal and said extension conductor is made of a second metal that is a different metal than said first metal.

19. The device of claim 1, wherein said neurological monitoring electrode is made of plastic coated with metal.

20. The device of claim 1, wherein said neurological monitoring electrode is made of plastic coated with a mixture of silver and silver chloride or of gold.
